(19) United States
(12) Patent Application Publication
(10) Pub. No.: US 2005/0148980 A1
(43) Pub. Date: Jul. 7, 2005

(54) Absorbent garment having outer shell and discreet absorbent assembly adapted for positioning therein

(75) Inventor: Matthew T. Fitton, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/750,381

(22) Filed: Dec. 30, 2003

(51) Int. Cl. A61F 13/15; A61F 13/20

(52) U.S. Cl. 604/385.3

(57) ABSTRACT

An absorbent garment having an outer shell and a discreet absorbent assembly adapted for positioning therein is disclosed. In particular embodiments, the absorbent garment comprises a garment shell and an inner absorbent assembly. The garment shell defines a waist opening and at least one leg opening, defines a body-side surface and an outward surface, and includes an elasticized shell waistband adapted to encircle the wearer. The inner absorbent assembly comprises an absorbent composite connected directly or indirectly to the garment shell. The inner absorbent assembly can include one or more posterior support straps which connect the absorbent composite to the garment shell. In certain embodiments, the absorbent assembly includes two posterior support straps which diverge from each other from the absorbent composite to the at garment shell. The absorbent assembly can also include an elasticized support waistband, and the absorbent composite can be connected to the elasticized support waistband. Also disclosed is a package including at least one garment shell and one or more absorbent assemblies.

### BACKGROUND OF THE INVENTION

This invention relates generally to absorbent garments, and more particularly to absorbent garments having the appearance of conventional clothing and having an outer shell and a discreet absorbent assembly adapted for positioning therein.

Personal wear garments and other articles find widespread use as personal care products including, without limitation, diapers, children's toilet training pants, disposable youth pants, adult incontinence garments, sanitary napkins and the like, as well as surgical bandages and sponges. The primary purpose of such articles is to take in and retain body exudates released by a wearer to thereby prevent soiling of the wearer's or caregiver's clothing. Certain absorbent articles are suitably partially or fully disposable in that they are intended to be discarded after a limited period of use, i.e., the articles are not intended to be laundered or otherwise restored for reuse.

In particular absorbent articles such as children's training pants, various attempts have been made to make the pants more visually appealing, such as by applying certain graphics or other features which make the pants appear more like conventional clothing, and more particularly like conventional undergarments. For example, training pants represent an intermediate stage for a child between using diapers and using cloth underpants. By making the training pants more closely resemble the undergarments or other clothing that an older sibling or parent wears, it is believed that children ready for toilet training will be more amenable to wearing the training pants. In another example, some children require the use of nighttime disposable absorbent pants to address bed-wetting problems (enuresis). Children requiring these absorbent pants generally desire the pants they are wearing to be as discreet as possible. Wearing an absorbent garment that resembles conventional clothing can be a significant benefit for such children. Additionally, a garment-like absorbent garment that minimized the likelihood that others would know that a child was wearing an enuresis pant, such as at a sleep-over party for children, would prevent embarrassment to the child suffering from enuresis.

One drawback to simply improving the external appearance of existing absorbent pants is that the entire pant must still be discarded after use. As a result, additional features which are added to entice children to wear the pants or otherwise conceal the absorbent look of the pants add further costs to making and using the pants. Moreover, clothes must still be worn over the absorbent pants, which can be uncomfortable and results in a rather bulky appearance. Also, to inhibit the leakage of exudates from absorbent articles such as training pants or other absorbent pants, it is important that the article fit generally snug against the wearer's body. For example, conventional training pants are constructed to provide a generally elastic fit about the wearer's waist and about the wearer's legs to inhibit leakage from the pants. However, many conventional garments that are worn about one's waist, such as certain styles of shorts, skirts, skorts, boxer shorts, swim trunks and the like, all have a more loose fitting appearance, particularly about the legs of the wearer. Also, it can in certain instances be useful to be able to remove and discard a wetted or soiled portion of a garment, such as an absorbent insert in an enuresis pant, but reuse a different portion of the garment, such as a garment-like shell.

Therefore, a need exists for an absorbent garment that resembles conventional clothing, that discreetly provides absorbent protection for discharges of bodily fluids, and that, in certain instances, includes portions that are removable, replaceable, and/or reusable.

### SUMMARY OF THE INVENTION

In response to the aforementioned needs and deficiencies in the art, a new absorbent garment has been invented.

In one embodiment, the present invention is directed to an absorbent garment comprising a garment shell and an inner absorbent assembly. The garment shell comprises a front panel assembly defining a front waist region, and a back panel-assembly defining a back waist region, the front panel assembly being connected to the back panel assembly so as to define a waist opening and at least one leg opening. The garment shell further comprise an elasticized shell waistband, the elasticized shell waistband adapted to encircle the wearer, the garment shell defining a body-side surface and an outward surface. The inner absorbent assembly comprises an absorbent composite which defines a front region and a back region. The inner absorbent assembly further comprises an elasticized support waistband defining a front waist section and a back waist section, the front waist section being connected to the absorbent composite front region. The inner absorbent assembly further comprises at least one posterior support strap connecting the elasticized support waistband to the absorbent composite, and the elasticized support waistband is connected to the body-side surface of the garment shell.

In another embodiment, the present invention is directed to an absorbent garment comprising a garment shell and an inner absorbent assembly. The garment shell comprises a front panel assembly defining a front waist region, and a back panel assembly defining a back waist region, the front panel assembly being connected to the back panel assembly so as to define a waist opening and at least one leg opening. The garment shell further comprises an elasticized shell waistband, the elasticized shell waistband adapted to encircle the wearer, the garment shell defining a body-side surface and an outward surface. The inner absorbent assembly comprises an absorbent composite which defines a front region and a back region, the front region being connected to the front waist region of the garment shell. The inner absorbent assembly further comprises two posterior support straps which connect the absorbent composite to at least one of the front waist region and the back waist region of the garment shell. The two posterior support straps diverge from each other from the absorbent composite to the at least one of the front waist region and the back waist region.

In still another embodiment, the present invention is directed to an absorbent garment comprising a garment shell and an inner absorbent assembly. The garment shell comprises a front panel assembly defining a front waist region, and a back panel assembly defining a back waist region, the front panel assembly being connected to the back panel assembly so as to define a waist opening and at least one leg opening. The garment shell further comprises an elasticized shell waistband, the elasticized shell waistband adapted to encircle the wearer, the garment shell defining a body-side surface and an outward surface. The inner absorbent assembly comprises an absorbent composite which defines a front region and a back region, the front region being connected to the front waist region of the garment shell. The inner absorbent assembly further comprises two posterior support straps which connect the front region of the absorbent composite to the back region of the absorbent composite. The two posterior support straps diverge from each other from the absorbent composite back region to the absorbent composite front region.

In yet another embodiment, the present invention is directed to an absorbent garment comprising a garment shell and an inner absorbent assembly. The garment shell comprises a front panel assembly defining a front waist region, and a back panel assembly defining a back waist region, the front panel assembly being connected to the back panel assembly so as to define a waist opening and at least one leg opening. The garment shell further comprises an elasticized shell waistband, the elasticized shell waistband adapted to encircle the wearer, the garment shell defining a body-side surface and an outward surface. The inner absorbent assembly comprises an absorbent composite which defines a front region and a back region, the front region being connected to the front waist region of the garment shell. The inner absorbent assembly further comprises at least one posterior support strap which connects the absorbent composite to the back waist region of the garment shell.

In another aspect, the present invention also pertains to a package comprising at least one garment shell and a plurality of inner absorbent assemblies. The at least one garment shell comprises a front panel assembly defining a front waist region, and a back panel assembly defining a back waist region, the front panel assembly being connected to the back panel assembly so as to define a waist opening and at least one leg opening. The at least one garment shell further comprises an elasticized shell waistband, the elasticized shell waistband adapted to encircle the wearer, the garment shell defining a body-side surface and an outward surface. Each inner absorbent assembly comprises an absorbent composite which defines a front region and a back region. Each inner absorbent assembly further comprises an elasticized support waistband defining a front waist section and a back waist section, the front waist section being connected to the absorbent composite front region. Each inner absorbent assembly further comprises at least one posterior support strap connecting the elasticized support waistband to the absorbent composite.

Definitions

Within the context of this specification, each term or phrase below will include the following meaning or meanings.

“Attached” refers to the joining, adhering, connecting, bonding, or the like, of two or more elements, either directly or indirectly by way of an intervening element or elements.

“Disposed,” “disposed on,” “disposed with,” “disposed at,” “disposed near” and variations thereof are intended to mean that one element can be integral with another element, or that one element can be a separate structure bonded to or placed with or placed near another element.

“Elastic,” “elasticized,” “elastomeric,” and “elasticity” mean that property of a material by virtue of which it tends to recover its original size and shape after removal of a force causing a deformation. As used to describe a component, “elastic” or “elasticized” means that the component can totally or partially exhibit that property. For example, the phrase “elasticized strip of material” can mean that the strip of material is elasticized over its entire length, width, or only a portion of one or both of its length and width.

“Hydrophilic” describes fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222. Surface Force Analyzer System, or a substantially equivalent system. When measured with this system, fibers having contact angles less than 90 degrees are designated “wettable” or hydrophilic, and fibers having contact angles greater than 90 degrees are designated “nonwettable” or hydrophobic.

“Layer” when used in the singular can have the dual meaning of a single element or a plurality of elements.

“Liquid impermeable,” when used in describing a layer or multi-layer laminate means that liquid body waste, such as urine, will not pass through the layer or laminate, under ordinary use conditions, in a direction generally perpendicular to the plane of the layer or laminate at the point of liquid contact.

“Liquid permeable” refers to any material that is not liquid impermeable.

“Longitudinal,” and “transverse” or “lateral,” have their customary meaning, in that the longitudinal axis is generally parallel to a vertical plane that bisects a standing wearer into left and right body halves when the article is worn, and the transverse or lateral axis lies in the plane of the article generally perpendicular to the longitudinal axis.

“Meltblown” refers to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameters. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface. Meltblown fibers used in the present invention are preferably substantially continuous in length.

The term “microfibers” means small-diameterfibers having an average diameter not greater than about 100 microns, for example, having a diameter of from about 0.5 microns to about 50 microns, more specifically microfibers may also have an average diameter of from about 1 micron to about 20 microns. Microfibers having an average diameter of about 3 microns or less are commonly referred to as ultra-fine microfibers. A description of an exemplary process of making ultra-fine microfibers may be found in, for example, U.S. Pat. No. 5,213,881, entitled “A Nonwoven Web With Improved Barrier Properties”.

“Non-woven” as used in reference to a material, web or fabric refers to such a material, web or fabric having a structure of individual fibers or threads that are interlaid, but not in a regular or identifiable manner as in a knitted fabric. Non-woven materials, fabrics or webs have been formed from many processes such as, for example, meltblowing processes, spunbonding processes, air laying processes, and bonded carded web processes. The basis weight of non-wovens is usually expressed in ounces of material per square yard (osy) or grams per square meter (gsm) and the fiber diameters are usually expressed in microns. (Note: to convert from osy to gsm, multiply osy by 33.91.).

“Operatively joined,” with reference to the attachment of an elastic member to another element, means that the elastic member when attached to or connected to the element, or treated with heat or chemicals, by stretching, mechanical straining or the like, gives the element elastic properties; and with reference to the attachment of a non-elastic member to another element, means that the member and element can be attached in any suitable manner that permits or allows them to perform the intended or described function of the joinder. The joining, attaching, connecting or the like can be either direct, such as by joining the member directly to an element, or can be indirect, such as by means of another member disposed between the member and the element.

“Refastenable” refers to the property of two elements being capable of releasable attachment, separation, and subsequent releasable reattachment without substantial permanent deformation or rupture.

“Releasably attached,” “releasably engaged” and variations thereof refer to two elements being connected or connectable such that the elements tend to remain connected absent a separation force applied to one or both of the elements, and the elements being capable of separation without substantial permanent deformation or rupture. The required separation force is typically beyond that encountered while wearing the article.

“Spunbonded fibers”, or “spunbond fibers”, means small-diameter fibers that are typically formed by extruding molten thermoplastic material as filaments from a plurality of fine capillaries of a spinneret having a circular or other configuration, with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, U.S. Pat. No. 3,502,538 to Petersen, and U.S. Pat. No. 3,542,615 to Dobo et al., each of which is incorporated by reference in its entirety and in a manner consistent with the present document. Spunbond fibers are quenched and generally not tacky when they are deposited onto a collecting surface. Spunbond fibers are generally continuous and often have average diameters larger than about 7 microns, and more particularly between about 10 and 30 microns. A spunbond material, layer, or substrate comprises spunbonded (or spunbond) fibers.

“Stretch bonded” refers to an elastic member being bonded to another member while the elastic member is extended at least about 25 percent of its relaxed length. More suitably, the term “stretch bonded” refers to the situation wherein the elastic member is extended at least about 100 percent, and even more suitably at least about 300 percent, of its relaxed length when it is bonded to the other member.

“Stretch bonded laminate” refers to a composite material having at least two layers in which one layer is a gatherable layer and the other layer is an elastic layer. The layers are joined together when the elastic layer is in an extended condition so that upon relaxing the layers, the gatherable layer is gathered.

“Thermoplastic” describes a material which softens when exposed to heat and which substantially returns to a non-softened condition when cooled to room temperature.

These terms may be further defined with additional language in the remaining portions of the specification.

### BRIEF DESCRIPTION OF THE DRAWINGS

FIG. 1 is a perspective view of one embodiment of the absorbent garment of the present invention;

FIG. 2 is a perspective view of another embodiment of the absorbent garment of the present invention, with portions cut away to show underlying features;

FIG. 3 is perspective view of one embodiment of an absorbent assembly of the absorbent garment of the present invention;

FIG. 4 is a perspective view of another embodiment of an absorbent assembly of the absorbent garment of the present invention;

FIG. 5 is a perspective view of yet another embodiment of an absorbent assembly of the absorbent garment of the present invention;

FIG. 6 is an elevated side view of another embodiment of the absorbent garment of the present invention, with a side seam of the absorbent garment shown in an unfastened condition, and with a portion cut away to show underlying features;

FIG. 7 is an elevated side view of another embodiment of the absorbent garment of the present invention, with a side seam of the absorbent garment shown in an unfastened condition, and with a portion cut away to show underlying features; and

FIG. 8 is an exploded perspective view of another embodiment of the absorbent garment of the present invention, with a portion cut away to show underlying features.

FIG. 9 is a perspective view of yet another embodiment of an absorbent assembly of the absorbent garment of the present invention;

FIG. 10 is a perspective view of yet another embodiment of an absorbent assembly of the absorbent garment of the present invention;

Corresponding reference characters indicate corresponding parts throughout the drawings.

### DETAILED DESCRIPTION

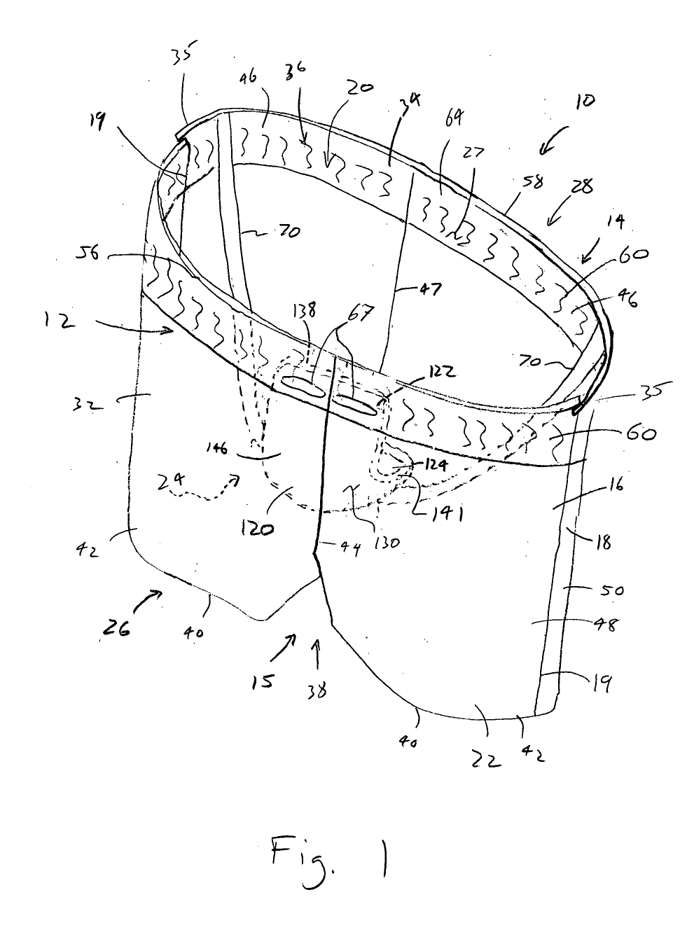

Referring now to the drawings, and in particular to FIGS. 1, 2, and 6-8, an absorbent garment according to various embodiments of the present invention is indicated in its entirety by the reference numeral 10. The absorbent garment 10 is configured to be worn on a wearer's waist and generally has a front waist region, indicated generally at 12, a back waist region, indicated generally at 14, and in particular embodiments and a crotch region, indicated generally at 15. The front and back waist regions 12, 14 have respective side margins 16, 18 which are in particular embodiments attached to each other along side seams 19 of the garment to form a three-dimensional configuration of the garment during wear and having a waist opening, generally indicated at 20. As used herein, the term “seam” is intended to refer to a region along which two components are overlapped or otherwise in abutment with each other and that are typically, though not necessarily, attached to each other.

As described further herein, the absorbent garment is suitably configured to resemble conventional clothing such as shorts (e.g., boxer shorts, gym shorts, running shorts, etc.), skirts, skorts (i.e., a combination of a skirt and a pair of shorts), swim trunks and the like, while providing the functions of conventional absorbent articles, such as taking in and retaining body exudates released by the wearer. The absorbent garment 10 comprises a garment shell, generally indicated at 22 and constructed to provide the desired resemblance of the garment to conventional clothing, and an inner absorbent assembly, generally indicated at 24, disposed within and adapted for positioning within the garment shell and constructed to take in and retain body exudates released by the wearer.

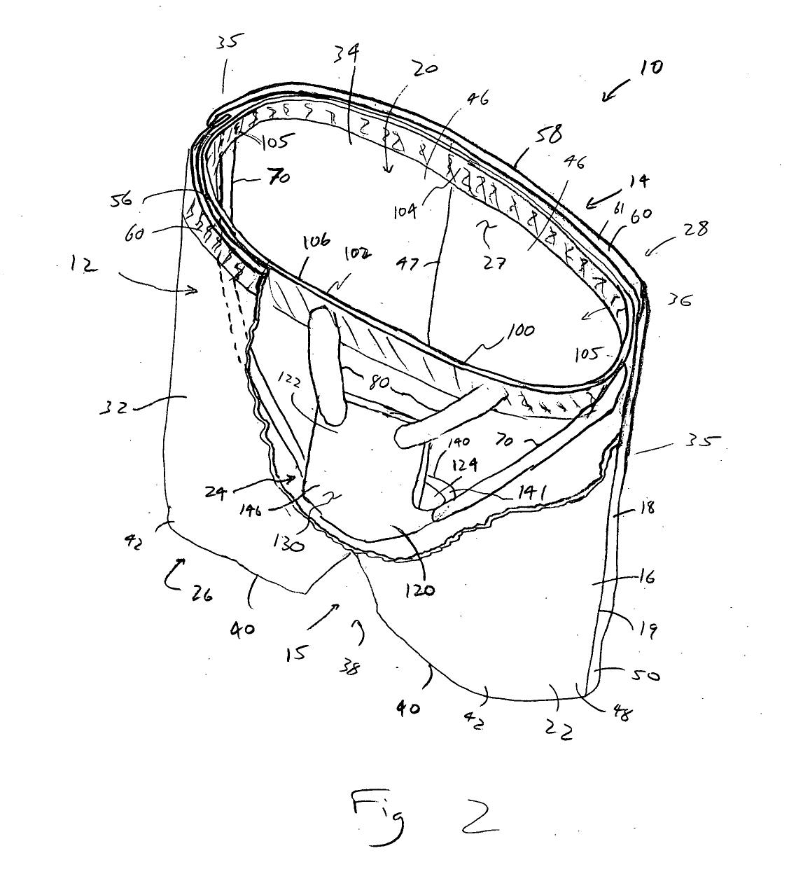

The garment shell 22 comprises a front panel assembly, which is generally indicated at 26, having laterally opposite side margins 48 and a back panel assembly, which is generally indicated at 28 in FIG. 2, having laterally opposite side margins 50. In the illustrated embodiment, the side margins 48 of the front panel assembly 26 broadly define the front side margins 16 of the absorbent garment 10 and the side margins 50 of the back panel assembly 28 broadly define the back side margins 18 of the absorbent garment. As will be described in further detail later herein, the side margins 48, 50 of the front and back panel assemblies 26, 28 of the garment shell 22 can be overlapped and in particular embodiments are attached to each other to broadly define the side seams 19 of the absorbent garment 10, and to define the three-dimensional configuration of the garment shell during wear.

In its three-dimensional configuration as shown in FIGS. 1, 2, and 6-8, the garment shell 22 has a front waist region 32 which at least in part defines the front waist region 12 of the absorbent garment 10, a back waist region 34 which at least in part defines the back waist region 14 of the absorbent garment, and front and back waist ends, designated 56 and 58, respectively, which together generally define a waist opening 36 of the garment shell. In the illustrated embodiment, the garment shell 22 is configured to resemble a pair of shorts and thus further has a crotch region 38 extending longitudinally between and interconnecting the front waist region 32 and the back waist region 34 of the garment shell. The crotch region 38 of the garment shell 22 at least in part defines the crotch region 15 of the absorbent garment 10, and also in part defines leg openings 40 of the garment shell (broadly referred to herein as outer leg openings of the absorbent garment). However, it is understood that the crotch region 38 of the garment shell 22 may be omitted (so that the crotch region 15 of the absorbent garment 10 is defined solely by the inner absorbent assembly 24 as described later herein), such as where the garment shell is intended to resemble a skirt (in which case only one leg opening 40 of the garment shell is provided to accommodate both legs of the wearer), without departing from the scope of this invention.

The front panel assembly 26 of the garment shell 22 comprises a pair of panel members 42 which are in particular embodiments permanently attached to each other, such as by ultrasonic bonding, pressure bonding, thermal bonding, adhesive bonding, stitching or other conventional attachment technique, along a central seam 44 extending longitudinally from the front waist region 32 to the crotch region 38 of the garment shell. The back panel assembly 28 comprises a pair of panel members 46 configured and permanently attached to each other in a manner similar to the panel members 42 of the front panel assembly 26 along a central seam 47 extending longitudinally from the back waist region 34 to the crotch region 38 of the garment shell 22. It is understood, however, that each of the front and back panel assemblies 26, 28 may be constructed of a single panel member (e.g., of unitary construction) without departing from the scope of this invention. Alternatively, the front and back panel members 42, 46 on one side of the garment shell 22 may be formed integrally at the crotch region 38 thereof so that no attachment of the panel members is necessary at the leg openings.

The panel members 42, 46 of the front and back panel assemblies 26, 28 of the garment shell 22 can be constructed of any suitable material, and more suitably a material that provides a generally cloth-like texture. The panel members 42, 46 are, in particular embodiments, constructed of a material which is relatively durable so that the garment shell 22 can be re-used through multiple replacements of the absorbent assembly. It is also contemplated that the panel members 42, 46 can, but need not necessarily be, constructed of a material suitable for laundering to permit laundering of the garment shell. In alternative embodiments, the garment shell is intended to be disposable after a single or several uses. The panel members 42, 46 may be constructed from natural and/or synthetic sources and may be include such materials as, for example, nonwovens such as spunbond, meltblown, spunbond film laminates, bonded carded web, spunlace, hydroentangled, and needlepunched fabrics; knit fabrics such as stretch knit, fleece knit, herringbone knit, jersey knit, raschel knit; and woven fabrics such as broadcloth, twill, percale, poplin, muslin, cambric, chino, flannel, silks and woolens. The panel members 42, 46 are suitably liquid permeable, although it is understood that the panel members may be liquid impermeable without departing from the scope of this invention.

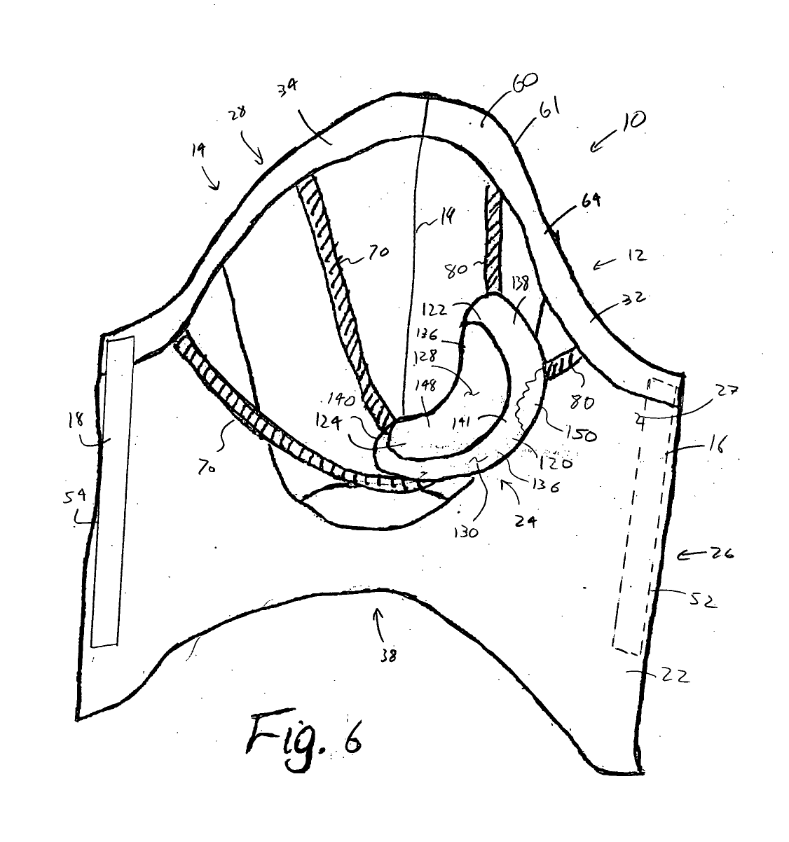

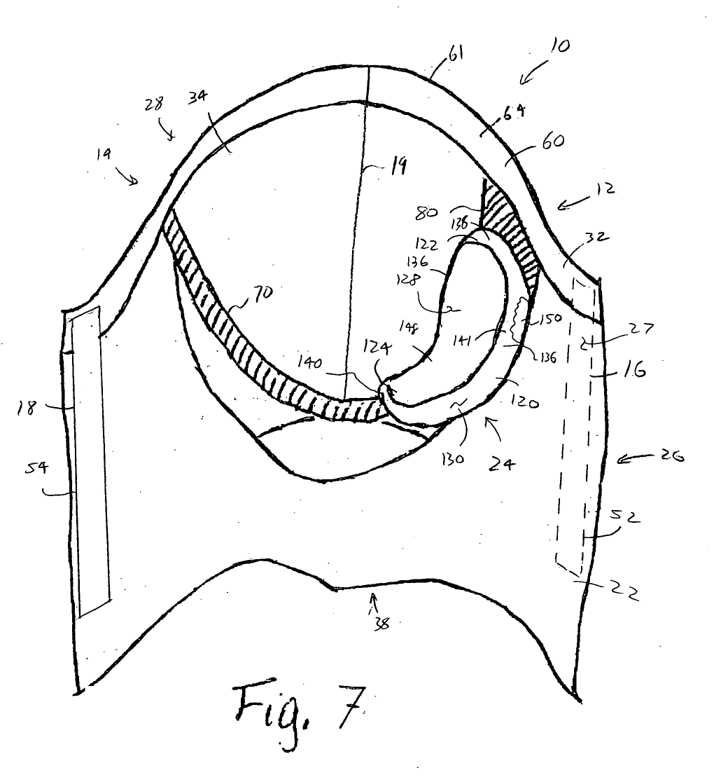

With particular reference to the embodiments representatively illustrated in FIGS. 6 and 7, the front and back panel assemblies 26, 28 of the garment shell 22 can be releasably attached to each other at the respective side margins 48, 50 of the panel assemblies. For example, in the illustrated embodiment, a fastening component 52 is attached to each side margin 48 of the front panel assembly 26 and is adapted for refastenable engagement with a complementary fastening component 54 attached to each respective side margin 50 of the back panel assembly 28. Although the garment shell 22 as illustrated in FIGS. 6 and 7 has the side margins 50 of the back panel assembly 28 overlapping the side margins 48 of the front panel assembly 26 upon releasable attachment, the garment shell can instead be configured so that the side margins of the front panel assembly overlap the side margins of the back panel assembly for releasable attachment.

The fastening components 52, 54 can comprise any refastenable fasteners suitable for garments, such as adhesive fasteners, cohesive fasteners, mechanical fasteners, or the like. In particularly suitable embodiments, the fastening components 52, 54 comprise mechanical fastening elements provided by interlocking geometric shaped materials such as hooks, loops, bulbs, mushrooms, arrowheads, balls on stems, male and female mating components, buckles, snaps, or the like. For example, in the illustrated embodiment the fastening components 52 comprise hook fasteners and the fastening components 54 comprise complementary loop fasteners arrayed so that the hook fasteners face generally away from the wearer. Alternatively, the fastening components 52 may comprise loop fasteners and the fastening components 54 may comprise complementary hook fasteners. One skilled in the art will recognize that the shape, density and polymer composition of the hooks and loops may be selected to obtain the desired level of engagement between the fastening components 52, 54. A more aggressive hook material may comprise a material with a greater average hook height and/or a greater percentage of directionally-aligned hooks. It is also contemplated that the fastening components 52, 54 can comprise interlocking similar surface fasteners, or adhesive and cohesive fastening elements such as an adhesive fastener and an adhesive-receptive landing zone or material; or the like.

“Loop fastener” as used herein refers to a fabric or material including a plurality of loop members. The loop material can be formed of any suitable material, such as acrylic, polyamide, polyethylene, polypropylene or polyester, and can be formed by methods such as warp knitting, stitch bonding or needle punching. Loop materials can also comprise any fibrous structure capable of entangling or catching hook materials, such as carded, spunbonded or other nonwoven webs or composites, including elastomeric and nonelastomeric composites. Suitable loop materials are available from Guilford Mills, Inc., Greensboro, N.C., U.S.A. under the trade designation No. 36549. Another suitable loop material can comprise a pattern un-bonded web as disclosed in co-assigned U.S. Pat. No. 5,858,515 issued Jan. 12, 1999 to Stokes, et al., which is incorporated herein by reference.

The loop material may be attached to a base, or backing structure, and the composite then attached to the particular component of the absorbent garment 10, such as the front or back panel assemblies 26, 28 of the garment shell 22, or the loop material may be attached directly to the absorbent garment component so that the component (e.g., the garment shell) serves as a backing for the loop material, or the loop material may be formed integrally with the component (e.g., the garment shell), such as by constructing one or more layers or surfaces of the component to comprise a loop material.

“Hook fastener” as used herein refers to a fabric or material having a base or backing structure and a plurality of hook members extending upwardly from at least one surface of the backing structure. It should be understood that the term “hook” as used in reference to the hook members is non-limiting in the sense that the engaging elements of the hook fasteners may comprise shapes such as hooks, “T's”, “mushrooms” or any other shape so long as they are adapted to releasably engage the loop fasteners so as to provide a secure, but non-destructive releasable attachment. It is understood that the attachment may be of limited lifetime; for example, gradual degradation of the attachment may occur with repeated engagements and disengagements.

In contrast to the loop fasteners which suitably comprise a flexible fabric, the hook material may advantageously comprise a resilient material to minimize unintentional disengagement of the fastening components 52, 54 as a result of the hook material becoming deformed and catching on clothing or other items. The term “resilient” as used in reference to the hook fasteners refers to an interlocking material having a predetermined shape and the property of the interlocking material to resume the predetermined shape after being engaged and disengaged from a mating, complementary interlocking material.

Suitable hook material can be molded or extruded from nylon, polypropylene, polyethylene or another suitable material. Suitable single-sided hook materials for the fastening components 52, 54 as well as other fastening components described later herein are available from commercial vendors such as Velcro Industries B.V., Amsterdam, Netherlands or affiliates thereof, identified as Velcro HTH-829, which has a thickness of about 0.9 millimeters (35 mils) and HTH-851, which has a thickness of about 0.5 millimeters (20 mils); and Minnesota Mining & Manufacturing Co., St. Paul, Minn. U.S.A., including specific materials identified as CS-600. As with the loop fastener, it is understood that the hook material may be formed integrally with a component of the absorbent garment 10, such as the garment shell 22 in the instance of the fastening components 52, 54, without departing from the scope of this invention.

The fastening components 52, 54 are shown in FIGS. 6 and 7 as having a generally rectangular shape, although they may instead be square, round, oval, curved or other suitable shapes. The fastening components 52, 54 extend along the respective side margins 48, 50 of the front and back panel assemblies 26, 28 generally from the waist ends 56, 58 of the panel assemblies to a position intermediate the waist ends and the leg openings 40 of the garment shell so that the absorbent garment side margins 16, 18 are releasably attached to each other along at least upper segments of the side seams 19. As an example, the fastening components 52, 54 suitably extend from the front and back waist ends 56, 58 of the garment shell 22 along the side margins 48, 50 thereof in the range of about 30 percent to about 90 percent of the length of the side margins (broadly, about 30 percent to about 90 percent of the length of the side seams 19 of the absorbent garment 10). However, it is understood that the fastening components 52, 54 may be longer or shorter without departing from the scope of this invention. Thus, in the illustrated embodiment, only a portion of the side seams 19 of the absorbent garment are releasably attached. The segment of the garment shell 22 along which the side margins 48, 50 are not releasably attached (e.g., extending from the bottom of the fastening components 52, 54 to the leg openings 40 of the garment shell) are suitably free from any form of attachment. In such an embodiment, the non-refastenable portion of the side seams 19 of the absorbent garment 10 are referred to as being open and the side margins 16, 18 thereof are referred to as being unattached. Alternatively, the side margins 48, 50 of the front and back assemblies 26, 28 of the garment shell 22 may be non-refastenably (e.g., frangibly or permanently) attached to each other along the portion of side margins extending from the bottom of the fastening components to the leg openings 40, such as by adhesive, by thermal, ultrasonic, or pressure bonding, or by other suitable attachment techniques.

It is also contemplated that the fastening components 52, 54 may instead extend from the leg openings 40 of the garment shell partially up along the side margins 48, 50 of the front and back panel assemblies 26, 28 (e.g., so that only a lower segment of the side seams 19 of the absorbent garment are refastenable). The side margins 48, 50 extending from the tops of the fastening components 52, 54 to the waist ends 56, 58 of the garment shell 22 may be non-refastenably (e.g., frangibly or permanently) attached to each other in the manner described previously. In other embodiments, the fastening components 52, 54 may extend the entire length of the side margins 48, 50 of the front and back panel assemblies 26, 28 of the garment shell 22 (e.g., such that the side seams 19 of the absorbent garment 10 are refastenable along their full length). Also, while the fastening components 52, 54 are illustrated as being continuous along each respective side margin 48, 50, it is understood that two or more fastening components may be attached to each respective side margin in spaced relationship along the side margin without departing from the scope of this invention.

As representatively illustrated in FIGS. 1, 2, and 8, it is further contemplated that the side margins 48, 50 of the garment shell 22 may instead be permanently or frangibly (e.g., non-refastenably) attached along all or part of the full length thereof whereby no portions of the side margins are refastenable. It is also understood that the garment shell 22 may be formed to omit the side margins 48, 50 thereof, such as by integrally forming the respective front and back panel members 42, 46 on each side of the shell.

The amount of overlap between the side margins 48, 50 of the front and back panel assemblies 26, 28 at the side seams 19 of the garment shell 22 (broadly, the overlap of the side margins 16, 18 of the front and back waist regions 12, 14 of the absorbent garment 10) is suitably in the range of about 0.1 inches (2.5 millimeters (mm)) to about 6 inches (152.4 mm), and more suitably in the range of about 0.5 (12.7 mm) inches to about 3 inches (76.2 mm). It is contemplated that in particular embodiments the fastening components 52, 54 on at least one of the front and back panel assemblies 26, 28 may have a width corresponding to the range of overlap to permit a variable fit of the absorbent garment over a relatively wide range of wearer sizes. It should be noted that the front and back panel assemblies 26, 28 can overlap at the side seams 19 in a “lap” seam manner as illustrated, or in a, “butt” or “fin” seam manner (not shown).

The fastening components 52, 54 are suitably attached to the respective front and back panel assemblies 26, 28 by mechanical bonding. As used herein, mechanical bonding refers to non-adhesive bonding, such as by the application of pressure, ultrasonic energy, heat, laser energy or any other suitable form of energy which joins the fastening components to the panel assemblies 26, 28. Alternatively, or additionally, the fastening components 52, 54 may be adhered, such as by adhesive or cohesive means, to the respective front and back panel assemblies 26, 28. It is also contemplated that the fastening components 52, 54 may be formed integrally with the respective front and back panel assemblies 26, 28 and remain within the scope of this invention.

In addition to the front and back panel assemblies 26, 28 of the garment shell 22 being, in particular embodiments, releasably attached to each other at the respective side margins 48, 50 thereof, or as an alternative thereto, it is contemplated that the panel assemblies may be releasably attached to each other at the crotch region 38 of the garment shell (not shown) to allow the garment shell to be unfastened at the crotch region and pulled up relative to the inner absorbent assembly 24 for inspecting or otherwise replacing the absorbent assembly. For example, fastening components may be attached to the front and back panel assemblies 26, 28 of the garment shell 22 generally at the crotch region 38 thereof to permit releasable attachment of the panel assemblies at the crotch region.

To further enhance the appearance of the absorbent garment 10 as well as the fit of the absorbent garment on the wearer's waist, one or more elastic members 64 (e.g., waistband elastics) can be operatively joined to the front and back panel assemblies 26, 28 generally at the respective waist ends 56, 58 thereof. For example, as representatively illustrated in FIGS. 1-2 and 6-8, an elastic member 64 can be operatively joined to the front waist end 56 of the garment shell 22. Another elastic member 64 can be operatively joined to the back waist end 58. The elastic members 64 can be operatively joined to the garment shell 22 while in a stretched condition so that upon retraction the elastic members gather the garment shell at the front and back waist ends 56, 58 to provide a gathered appearance and to further provide an elastic fit of the absorbent garment on the wearer's waist. Alternatively, it is contemplated that multiple elastic members (not shown) may be attached to each waist end 56, 58 of the garment shell 22 and extend laterally across all or only a portion of the width of the respective waist end without departing from the scope of this invention.

With further reference to FIGS. 1-8, the inner absorbent assembly 24 comprises an absorbent composite 120. The absorbent composite 120 includes a front region 122, a back region 124, a body-side surface 128 configured for contiguous relationship with the wearer, and an outer surface 130 opposite the body-side surface. The front region 122 comprises the portion of the absorbent composite 120 which, when the absorbent garment 10 is worn, is disposed toward the front waist region 32 of the garment shell 22, while the back region 124 comprises the portion of the absorbent composite 120 which is disposed toward the crotch region 38 and/or the back waist region 34 of the garment shell 22. With additional reference to FIGS. 5-7, the absorbent composite 120 also has laterally opposite side edges 136 and longitudinally opposite ends, respectively designated herein as front end 138 and back end 140.

The absorbent composite 120 is suitably “disposable,” which as used herein refers to components that are intended to be discarded after a limited period of use instead of being laundered or otherwise restored for reuse. However, it is contemplated that the absorbent composite may be re-useable and remain within the scope of this invention. By way of illustration only, various materials and methods for constructing the absorbent composite 24 are disclosed in PCT Patent Application WO 00/37009 published Jun. 29, 2000 by A. Fletcher et al; U.S. Pat. No. 4,940,464 issued Jul. 10, 1990 to Van Gompel et al.; and U.S. Pat. No. 5,766,389 issued Jun. 16, 1998 to Brandon et al., which are incorporated herein by reference.

Referring to FIG. 6-8, the absorbent composite 120 in particular embodiments comprises an outer cover 146, a bodyside liner 148 in superposed relationship with the outer cover, an absorbent body 150 disposed between the outer cover and the bodyside liner, and a pair of laterally spaced containment flaps (not shown) configured to inhibit the transverse flow of body exudates on the liner to the side edges 136 of the absorbent assembly.

The outer cover 146 of the absorbent composite 120 can, but need not, comprise a material which is substantially liquid impermeable, and can be stretchable or non-stretchable. As used herein, the term “stretchable” refers to a material that may be extensible or elastic. That is, the material may be extended, deformed or the like, without breaking, and may or may not significantly retract after removal of an extending force. The term “extensible” refers to that property of a material where upon removal of an elongating force, the material experiences a substantially permanent deformation, or the material does not exhibit a significant retractive force.

More suitably, the outer cover 146 comprises a multi-layered laminate structure (not shown) in which at least one of the layers is liquid impermeable. For instance, the outer cover 146 can include a liquid permeable outer layer and a liquid impermeable inner layer which are suitably joined together by a laminate adhesive, ultrasonic bonds, pressure bonds, thermal bonds, or the like. Suitable laminate adhesives, which can be applied continuously or intermittently as beads, a spray, parallel swirls, or the like, can be obtained from Findley Adhesives, Inc., of Wauwatosa, Wis., U.S.A., or from National Starch and Chemical Company, Bridgewater, N.J., U.S.A. The liquid permeable outer layer can be any suitable material and is desirably one that provides a generally cloth-like texture and appearance. One example of such a material is a 20 gsm (grams per square meter) spunbond polypropylene nonwoven web. The outer layer may also be made of those materials described later herein from which the liquid permeable bodyside liner 148 is made.

In a multi-layered laminate outer cover 146, the inner layer of the outer cover 146 can be both liquid and vapor impermeable, or it may be liquid impermeable and vapor permeable. The inner layer can be manufactured from a thin plastic film, although other flexible liquid impermeable materials may also be used. The liquid impermeable inner layer can prevent waste material from wetting articles, such as bed sheets and clothing, as well as the wearer and caregiver. A suitable liquid impermeable film for use as a liquid impermeable inner layer of the outer cover 146 is a 0.02 millimeter polyethylene film commercially available from Pliant Corporation of Schaumburg, Ill., U.S.A.

Alternatively, the outer cover 146 may comprise a single layer of liquid impermeable material. As earlier mentioned, the liquid impermeable material can permit vapors to escape from the interior of the disposable absorbent article, while still preventing liquids from passing through the outer cover 146. For example, the outer cover 146 may be constructed of a microporous polymer film or a nonwoven fabric that has been coated or otherwise treated to impart a desired level of liquid impermeability. One such microporous film is a PMP-1 film material commercially available from Mitsui Toatsu Chemicals, Inc., Tokyo, Japan, or an XKO-8044 polyolefin film commercially available from 3M Company, Minneapolis, Minn. U.S.A. The single layer outer cover 146 may also be embossed and/or matte finished to provide a more cloth-like appearance.

The liquid permeable bodyside liner 148 is illustrated as overlying the outer cover 146 and absorbent body 150, and may but need not have the same dimensions as the outer cover 146. The bodyside liner 148 is desirably compliant, soft feeling, and non-irritating to the wearer's skin. Further, the bodyside liner 148 can be less hydrophilic than the absorbent body 150, to present a relatively dry surface to the wearer and to permit liquid to readily penetrate through the liner. Alternatively, the bodyside liner 148 can be more hydrophilic or can have essentially the same affinity for moisture as the absorbent body 150 to present a relatively wet surface to the wearer to increase the sensation of being wet. This wet sensation can be useful as a training aid. The hydrophilic/hydrophobic properties can be varied across the length, width and depth of the bodyside liner 148 and absorbent body 150 to achieve the desired wetness sensation or leakage performance.

The bodyside liner 148 can be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and nonwoven fabrics can be used for the bodyside liner 148. For example, the bodyside liner 148 can be composed of a meltblown or spunbonded web of polyolefin fibers. The bodyside liner can also be a bonded-carded web composed of natural and/or synthetic fibers. The bodyside liner 148 can be composed of a substantially hydrophobic material, and the hydrophobic material can, optionally, be treated with a surfactant or otherwise processed to impart a desired level of wettability and hydrophilicity. For example, the material can be surface treated with about 0.45 weight percent of a surfactant mixture comprising Ahcovel N-62 from Hodgson Textile Chemicals of Mount Holly, N.C., U.S.A. and Glucopan 220UP from Henkel Corporation of Ambler, Pennsylvania in an active ratio of 3:1. The surfactant can be applied by any conventional means, such as spraying, printing, brush coating or the like. The surfactant can be applied to the entire bodyside liner 148 or can be selectively applied to particular sections of the bodyside liner, such as the medial section along the longitudinal center line.

One example of a suitable liquid permeable bodyside liner 148 is a nonwoven bicomponent web having a basis weight of about 27 gsm. The nonwoven bicomponent web can be a spunbond bicomponent web, or a bonded carded bicomponent web. Suitable bicomponent fibers include a polyethylene/polypropylene bicomponent fiber available from CHISSO Corporation, Osaka, Japan. In this particular bicomponent fiber, the polypropylene forms the core and the polyethylene forms the sheath of the fiber. Other fiber orientations are possible, such as multi-lobe, side-by-side, end-to-end, or the like.

The absorbent body 150 is positioned between the outer cover 146 and the bodyside liner 148, which can be joined together by any suitable means such as adhesives, ultrasonic bonds, pressure bonds, thermal bonds, or the like. The absorbent body 150 can be any structure which is generally compressible, conformable, non-irritating to the child's skin, and capable of absorbing and retaining liquids and certain body wastes, and may be manufactured in a wide variety of sizes and shapes, and from a wide variety of liquid absorbent materials commonly used in the art. For example, the absorbent body 150 can suitably comprise a matrix of hydrophilic fibers, such as a web of cellulosic fluff, mixed with particles of a high-absorbency material commonly known as superabsorbent material. In a particular embodiment, the absorbent body 150 comprises a matrix of cellulosic fluff, such as wood pulp fluff, and superabsorbent hydrogel-forming particles. The wood pulp fluff can be exchanged with synthetic, polymeric, meltblown fibers or short cut homofil bicomponent synthetic fibers and natural fibers. The superabsorbent particles can be substantially homogeneously mixed with the hydrophilic fibers or can be nonuniformly mixed. The fluff and superabsorbent particles can also be selectively placed into desired zones of the absorbent body 150 to better contain and absorb body exudates. The concentration of the superabsorbent particles can also vary through the thickness of the absorbent body 150. Alternatively, the absorbent body 150 can comprise a laminate of fibrous webs and superabsorbent material, a foam or other suitable web construction.

Suitable superabsorbent materials can be selected from natural, synthetic, and modified natural polymers and materials. The superabsorbent materials can be inorganic materials, such as silica gels, or organic compounds, such as crosslinked polymers, for example, sodium neutralized polyacrylic acid. Suitable superabsorbent materials are available from various commercial vendors, such as BASF Corporation, Charlotte, N.C., U.S.A., and Stockhausen GmbH & Co. KG, D-47805. Krefeld, Federal Republic of Germany. Typically, a superabsorbent material is capable of absorbing at least about 10 times its weight in water, and suitably is capable of absorbing more than about 25 times its weight in water.

In one embodiment, the absorbent body 150 comprises a blend of wood pulp fluff and superabsorbent material. One suitable type of pulp is identified with the trade designation CR1654, available from U.S. Alliance, Childersburg, Ala., U.S.A., and is a bleached, highly absorbent sulfate wood pulp containing primarily soft wood fibers and about 16 percent hardwood fibers. In general, the superabsorbent material is present in the absorbent body 150 in an amount of from 0 to about 90 weight percent based on total weight of the absorbent assembly. The absorbent body 150 may or may not be wrapped or encompassed by a suitable wrap, such as a meltblown wrap or cellulosic tissue wrap, that aids in maintaining the integrity and/or shape of the absorbent assembly during use.

The absorbent composite 120 is illustrated in the Figures as having a generally cup-like shape. A cup-like shape can, in particular embodiments, allow the absorbent composite to be relatively small and discreet while worn, but still provide the wearer with adequate absorbency and leakage protection. For example, in particular embodiments, the absorbent composite side edges 136, front end 138, and back end 140 are urged upward generally toward the wearer to define an upwardly urged periphery 141 of the absorbent composite. An upwardly urged periphery is any portion of an absorbent composite that, when the absorbent composite is positioned against a wearer, sits 0.5 cm or more closer to the wearer than a point on the body-side surface 128 of the absorbent composite 120 positioned furthest from the wearer's body during donning. The upwardly urged periphery 141 can provide additional gasketing effect and thus minimize leakage during wear, despite that fact that, in particular embodiments such as enuresis garments, the absorbent composite 120 is relatively small in size as compared to conventional absorbent composites disposed within such garments. In particular embodiments, the absorbent composite 120 is constructed to provide an particular absorbent capacity, and the upwardly urged periphery 141 of the absorbent composite comprises absorbent material which provides at least about 10%, more particularly at least about 20%, and still more particularly at least about 30% of the absorbent capacity of the absorbent composite. For example, a portion of the absorbent body 150, such as wood pulp fluff and/or superabsorbent material, can extend into the upwardly urged periphery 141 of the absorbent composite 120. It is understood that, although the Figures representatively illustrate the absorbent composite 120 as assuming a cup-like shape, the absorbent composite may be other than cup-shaped, such as flat or V-shaped, and may be rectangular, hourglass-shaped, T-shaped, 1-shaped or other suitable shape without departing from the scope of this invention.

The containment flaps (not shown) are in particular embodiments located generally adjacent to the side edges 136 of the absorbent composite 120, and can extend longitudinally along the entire length of the absorbent composite or only partially along the length of the absorbent composite. Flap elastic members (not shown) can be operatively joined with the containment flaps in a suitable manner as is well known in the art, such as by adhering the elastic members to the flaps while the elastic members are in a stretched condition so that the flaps are biased by the elastic members to a longitudinally gathered configuration. The elasticized containment flaps can define a partially unattached distal edge (not shown), unattached to the liner 148, which assumes an upright configuration in at least a portion of the absorbent composite 120 during wear to form a seal (e.g., an elastic fit) against the wearer's body. The containment flaps can in particular embodiments extend laterally across the absorbent composite 120, and be located generally adjacent to the front and back ends 138, 140 of the absorbent composite 120. Suitable constructions and arrangements for the containment flaps are generally well known to those skilled in the art and are described in U.S. Pat. No. 4,704,116 issued Nov. 3, 1987 to Enloe, which is incorporated herein by reference to the extent consistent herewith. It is understood, however, that the containment flaps may be omitted without departing from the scope of this invention.

The flap elastic members and the waist elastic members 64 operatively joined with the garment shell can be formed of any suitable elastic material. As is well known to those skilled in the art, examples of suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat, such that elastic retractive forces are imparted to the substrate.

The absorbent composite 120 can also incorporate other materials or components designed primarily to receive, temporarily store, and/or transport liquid along the mutually facing surface with the absorbent body 150, thereby maximizing the absorbent capacity of the absorbent composite. For example, one suitable additional component is commonly referred to as a surge layer (not shown). Surge layers are generally well known in the art as being constructed to quickly collect and temporarily hold liquid surges, and to transport the temporarily held liquid to the absorbent body 150.

Various woven and non-woven fabrics can be used to construct the surge layer. For example, the surge layer may be a layer made of a meltblown or spunbond web of synthetic fibers, such as polyolefin fibers. The surge layer may also be a bonded-carded-web or an airlaid web composed of natural and synthetic fibers. The bonded-carded-web may, for example, be a thermally bonded web that is bonded using low melt binder fibers, powder or adhesive. The webs can optionally include a mixture of different fibers. The surge layer may be composed of a substantially hydrophobic material, and the hydrophobic material may optionally be treated with a surfactant or otherwise processed to impart a desired level of wettability and hydrophilicity.

Examples of materials suitable for the surge layer are set forth in U.S. Pat. No. 5,486,166 issued Jan. 23, 1996 in the name of C. Ellis et al.; U.S. Pat. No. 5,490,846 issued Feb. 13, 1996 in the name of Ellis et al.; and U.S. Pat. No. 5,364,382 issued Nov. 15, 1994 in the name of Latimer et al., the disclosures of which are hereby incorporated by reference in a manner consistent with the present document.

The absorbent composite 120 is in particular embodiments connected to the garment shell 22. As will be described in more detail, the absorbent composite 120 can be permanently attached, removably attached, or refastenably attached to the garment shell 22. Further, the absorbent composite 120 can be attached directly to the garment shell 22, or indirectly by way of an intervening element or elements, such as, in particular embodiments which shall be described, one or more support straps.

In certain embodiments, the absorbent composite 120 is connected directly to the garment shell 22. For example, as representatively illustrated in FIG. 1, the front region 122 of the absorbent composite 120 is attached to the front waist region 32 of the garment shell 22 at attachment regions 67. For example, a portion of the outer cover 146, the liner 148, or both can be attached to the front waist region 32 of the garment shell 22. The absorbent composite 120 can be permanently attached to the garment shell 22, such as by adhesive, by thermal, pressure, or ultrasonic bonding, or by other suitable attachment technique. Alternatively, the absorbent composite 120 can be releasably attached to the garment shell 22, such that the absorbent composite 120 can be removed after soiling. In certain embodiments, the absorbent composite 120 includes a frangible line of weakness (not shown), such as a row of perforations in one or both of the outer cover 146 and the liner 148. In yet additional embodiments, the absorbent composite 120 can be refastenably attached, such as by fastening components attached to the inner surface of the garment shell 22 generally at the front waist end 56 thereof (not shown). For example, loop fasteners can be disposed on the inner surface of the garment shell 22, and the absorbent composite 120 can have disposed thereon a suitable hook material for releasable attachment to the loop fasteners.

The absorbent assembly 24 further includes at least one posterior support strap 70. “Posterior support strap” as used herein means a component that assists in supporting the absorbent composite 120 against the wearer within the garment shell 22, and that is configured to be at least partially disposed toward the posterior of a wearer. The posterior support strap 70, in certain embodiments, connects the absorbent composite 120 to the garment shell 22. For example, as representatively illustrated in FIG. 7, a posterior support strap 70 can extend from the absorbent composite 120 to the back waist region 34 of the garment shell 22. In a particular embodiment, the posterior support strap is connected to the absorbent composite back end 140 (FIG. 7). The posterior support strap 70 can be wider than, narrower than, or the same width as the absorbent composite 120. In particular embodiments, the posterior support strap 70 is narrower than the absorbent composite 120 to enhance discretion and reduce material cost. In an alternative embodiment, the width of the at least one posterior support strap 70 increases as it travels in a direction from the absorbent composite 120 to the back waist region 34 of the garment shell 22. Such a configuration can provide added comfort to the wearer. For example, the width of a posterior support strap 70 can increase at least about twofold, more particularly at least about threefold, and still more particularly at least about fivefold as it travels in a direction from the absorbent composite 120 to the back waist region 34 of the garment shell 22.

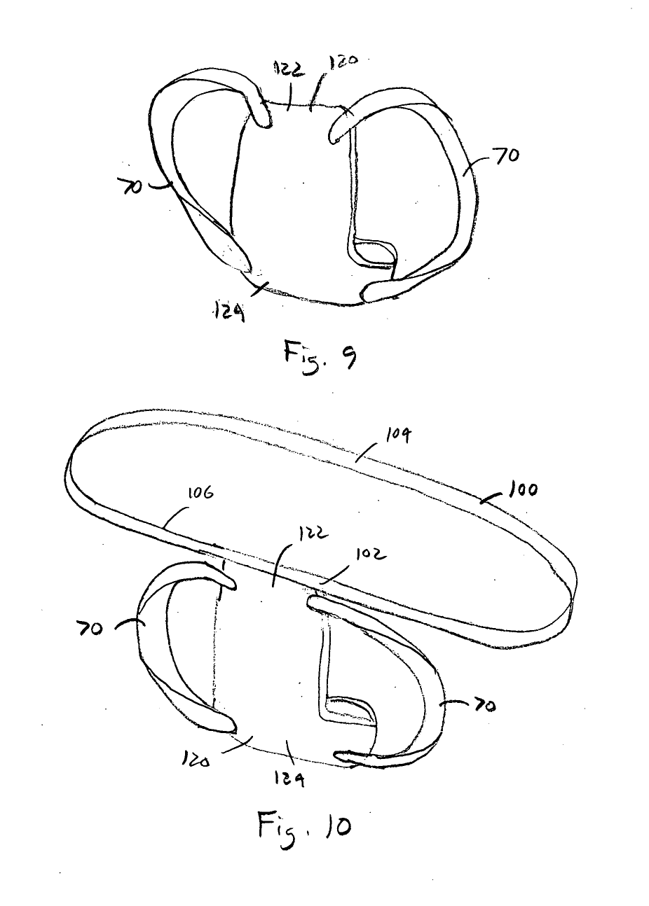

In alternative embodiments, the absorbent assembly 24 can include two posterior support straps 70. For example, as representatively illustrated in FIGS. 1 and 6, two posterior support straps 70 can connect the absorbent composite 120 to the front waist region 32, the back waist region 34, or both the front and back waist regions 32, 34 at side waist regions 35 (generally, the regions where the front waist region 32 meets the back waist region 34) of the garment shell 22. In particular embodiments, representatively illustrated in FIGS. 1 and 6, the two posterior support straps 70 diverge from each other as they travel in a direction from the absorbent composite 120 to the side waist regions 35. In such an embodiment, the two posterior support straps 70 can, but need not, connect the back region 124 of the absorbent composite 120 to the front region 122 of the absorbent composite 120 (representatively illustrated in FIG. 9).

In particular embodiments, the absorbent assembly 23 further includes at least one anterior support strap 80. “Anterior support strap” as used herein means a component that assists in supporting the absorbent composite 120 against the wearer within the garment shell 22, and that is configured to be disposed toward the anterior of a wearer. The anterior support strap 80, in certain embodiments, connects the absorbent composite 120 to the garment shell 22. For example, as representatively illustrated in FIG. 7, an anterior support strap 80 can extend from the absorbent composite front region 122 to the front waist region 32 of the garment shell 22. In a particular embodiment, the anterior support strap 80 is connected to the absorbent composite front end 138 (FIG. 7). The anterior support strap 80 can be wider than, narrower than, or the same width as the absorbent composite 120. In particular embodiments, the anterior support strap 80 is narrower than the absorbent composite 120 to enhance discretion and reduce material cost.

In alternative embodiments, the absorbent assembly 24 can include two anterior support straps 80. For example, as representatively illustrated in FIG. 6, two anterior support straps 80 can connect the absorbent composite 120 to the front waist region 32, the back waist region 34, or both the front and back waist regions 32, 34 at side waist regions 35 of the garment shell 22. In particular embodiments, representatively illustrated in FIG. 6, two anterior support straps 80 diverge from each other as they travel in a direction from the absorbent composite 120 to the front waist regions 32 of the garment shell 22.

In particular embodiments, the support straps 70, 80 can be, but need not be, stretchable, and more suitably elastic. For example, the support straps 70, 80 can be stretchable to a length in the range of about 5 percent to about 75 percent of their unstretched length. The support straps, 70, 80 are suitably constructed of elastomeric materials, including but not limited to elastic strands, elastic films, and nonwoven elastic webs such as meltblown or spunbond elastomeric fibrous webs. Examples of suitable elastomeric materials include ESTANE® elastomeric polyurethanes (available from B.F. Goodrich and Company located in Cleveland, Ohio), PEBAX® elastomers (available from AtoChem located in Philadelphia, Pa.), HYTREL® elastomeric polyester (available from E.I. DuPont de Nemours located in Wilmington Del.), KRATON® elastomer (available from Kraton Polymers, Inc. of Houston, Tex.), strands of LYCRA® elastomer (available from E.I. DuPont de Nemours located in Wilmington Del.) or the like, as well as combinations thereof. Suitable elastomeric materials may be braided, knit, woven or otherwise combined with natural fibers, or synthetic fibers such as polyester, nylon or polyolefins. Additional examples include stretch-bonded laminates and neck-bonded laminates. “Neck-bonded laminate” refers to a composite material having an elastic member that is bonded to another member while that member is extended in the machine direction, creating a necked material that is elastic in the cross-direction. Examples of neck-bonded laminates are disclosed in U.S. Pat. Nos. 4,965,122; 4,981,747; 5,226,992; and 5,336,545, which are incorporated herein by reference to the extent consistent herewith. In particular embodiments, the support straps 70, 80 comprise an elastomeric nonwoven material. Alternatively, one or more of the support straps 70, 80 may be constructed of an extensible material, or may be constructed of a non-stretchable material, without departing from the scope of this invention.

The support straps 70, 80 can be attached to the absorbent composite 120, such as to the bodyside liner 148 and/or to the outer cover 146, by adhesive, or by thermal, pressure, or ultrasonic bonding, or by other suitable attachment techniques known to those skilled in the art. Alternatively, the support straps 70, 80 can be formed as an integral portion of a component of the absorbent composite 120. In yet another alternative, one or more of the support straps 70, 80 can be releasably or refastenably attached to the absorbent composite 120, such as via cooperating hook and loop fastening components similar to those described earlier.

The support straps 70, 80 can be attached to the garment shell 22 by adhesive, or by thermal, pressure, or ultrasonic bonding, or by other suitable attachment techniques known to those skilled in the art. Alternatively, the support straps 70, 80 can be formed as an integral portion of a component of the garment shell 22. In yet another alternative, one or more of the support straps 70, 80 can be releasably or refastenably attached to the garment shell 22, such as via cooperating hook and loop fastening components similar to those described earlier. In particular embodiments, one or more of the support straps 70, 80 include a frangible line of weakness (not shown), such as perforations, to allow a user to tearably remove the absorbent composite 120 from the absorbent garment 10, such as after the release of bodily fluids.

If one or more of the support straps 70, 80 are attached to the absorbent composite 120, the garment shell 22, or both via hook-and-loop type fastening components, such fastening components may be attached to the support straps 70, 80, the absorbent composite 120, or the garment shell 22 by adhesive, by thermal, pressure, or ultrasonic bonding, or by any other suitable attachment technique. It is also contemplated that the fastening components may be formed integrally with any of these components of the absorbent garment 10.

Referring to FIGS. 2-5, 8, and 10, the absorbent assembly 24, in particular embodiments, further comprises an elasticized support waistband 100. As explained below in more detail, an elasticized support waistband 100 can have connected thereto the absorbent composite 120, as well as one or more support members 70, 80. An elasticized support waistband 100, combined with an absorbent composite 120 and one or more support members 70, 80, can constitute an absorbent assembly 24 that can be completely removed from the article after being insulted with a bodily fluid, providing discreet and convenient incontinence protection in the absorbent garment 10. For example, a young child suffering from enuresis can wear the garment-like absorbent garment 10 to a friend's house for a sleepover event, and in the morning remove and dispose of the absorbent assembly comprised of an elasticized support waistband 100, absorbent composite 120, and one or more support straps 70, 80, leaving only the garment shell 22 on his person. In this way, the likelihood that other children or adults will know that the child requires an enuresis garment is minimized.

The elasticized support waistband 100 defines a front waist section 102 and a back waist section 104, and is adapted for connection to the garment shell 22. The elasticized support waistband 100 can partially encircle the wearer, or can fully encircle the wearer. In particular embodiments, the elasticized support waistband 100 is connected to the body-side surface 27 of the garment shell 22. For example, the elasticized support waistband front waist section 102 can be connected to the garment shell front waist region 32, or the elasticized support waistband back waist section 104 can be connected to the garment shell back waist region 34, or both. In particular embodiments, the elasticized support waistband front waist section 102 is connected to the garment shell front waist end 56. In such an embodiment, the elasticized support waistband back waist section 104 can, but need not be, connected to the garment shell back waist end 58.

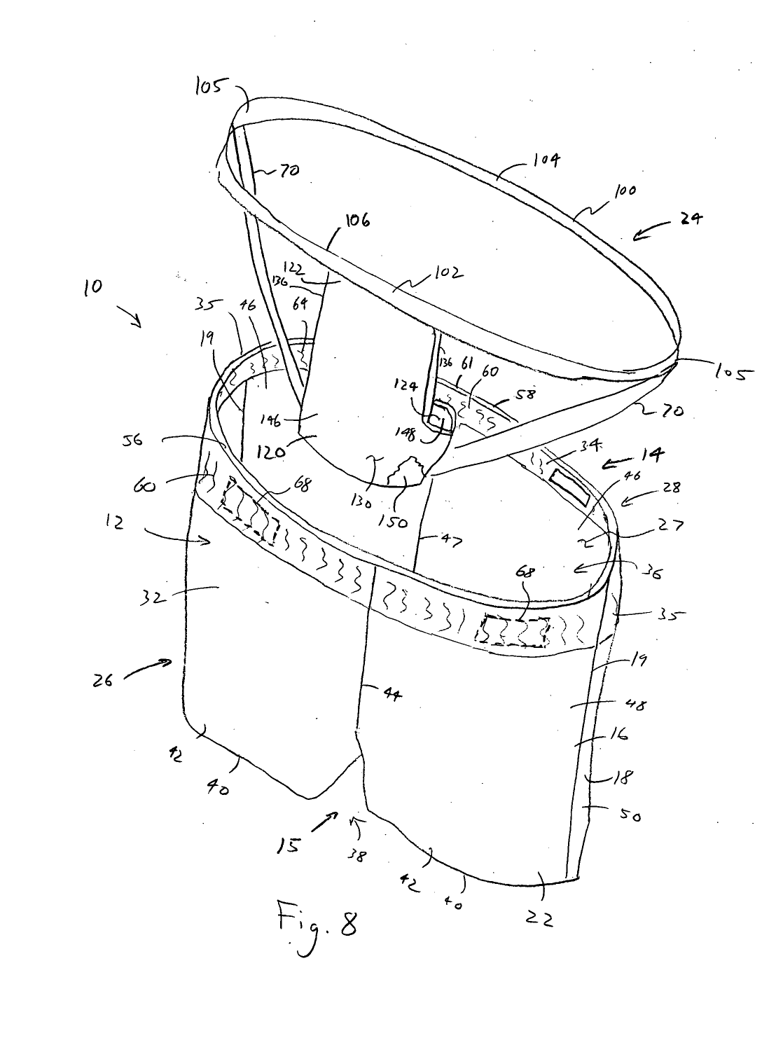

The elasticized support waistband 100 can be permanently, releasably, or refastenably connected to the garment shell 22. In certain embodiments, the elasticized support waistband 100 is releasably attached to the garment shell 22, such as by adhesive, or by thermal, pressure, or ultrasonic bonding, adapted to connect to elasticized support waistband 100 to the garment shell 22 at a level of strength consistent with the previously articulated definition of “releasably attached.” Alternatively, the elasticized support waistband 100 can be refastenably attached to the garment shell 22, such as by fastening components 68 attached to the inner surface of the garment shell generally at, for example, the front waist end 56 thereof (FIG. 8). For example, hook fasteners can be attached to the inner surface of the garment shell 22, and the elasticized support waistband 100 can provide a suitable loop material for releasable attachment to the hook fasteners.

In certain embodiments, the elasticized support waistband 100 is connected to the garment shell 22 in a manner such that the elasticized support waistband remains connected to the garment shell 22 during donning of the absorbent garment 10, but becomes partially unattached or completely unattached to the garment shell 22 after being worn for some time. In such an embodiment, an absorbent assembly 24 having an elasticized support waistband 100 and one or more support straps is conveniently held in a three-dimensional configuration during donning, increasing the ease with which a wearer can insert his legs and pull the garment up around the waist. Subsequent to donning, the elasticized support waistband 100 can become partially or completely unconnected to the garment shell, allowing the inner absorbent assembly 24 to move independently of the garment shell 22, and better conform to the body to enhance the leakage control and absorptive qualities of the absorbent composite 120. One example of a bonding technique which can provide such a feature is a heat sensitive adhesive, constructed to provide an effective bond between exemplary absorbent garment materials (such as polymer-based nonwoven webs) at room temperature (about 60-70 degrees Fahrenheit), but become weakened at temperatures near the normal human body temperature (above about 95 degrees Fahrenheit). Additional examples of suitable bonding techniques for such a feature are pressure bonds or ultrasonic bonds. In another embodiment (not shown), the elasticized support waistband 100 can be integrally formed with but longitudinally offset from the elasticized shell waistband 60, and a region longitudinally between the elasticized shell waistband and the elasticized support waistband can include a frangible line of weakness constructed to break or to be broken during wearing of the garment.

In those embodiments of the absorbent garment of the present invention that include an elasticized support waistband 100, the absorbent composite 120 can, in particular embodiments, be connected directly to the elasticized support waistband 100. For example, as representatively illustrated in FIGS. 5 and 8, the front region 122 of the absorbent composite 120 is attached to the front waist section 102 of the elasticized support waistband 100. For example, a portion of the outer cover 146, the liner 148, or both can be attached to the front waist section 102 of the elasticized support waistband 100. The absorbent composite 120 can be permanently or releasably attached to the elasticized support waistband 100, such as by adhesive, by thermal, pressure, or ultrasonic bonding, or by other suitable attachment technique. In particular embodiments, the absorbent composite 120 is releasably attached to the elasticized support waistband 100, such that the absorbent composite 120 can be removed after soiling and the elasticized support waistband 100 reused. In yet additional embodiments, the absorbent composite 120 can be refastenably attached, such as by complementary fastening components disposed on both the absorbent composite 120 and the elasticized support waistband 100, such as hook and loop fasteners.

In those embodiments of the absorbent garment of the present invention that include an elasticized support waistband 100, and that further include one or more support straps 70, 80 as previously described, the support straps 70, 80 are suitably attached to the elasticized support waistband 100. Such attachment of the support straps 70, 80 to the elasticized support waistband 100 can be by adhesive, or by thermal, pressure, or ultrasonic bonding, or by other suitable attachment techniques known to those skilled in the art. Alternatively, the support straps 70, 80 can be formed as an integral portion of the elasticized support waistband 100. In yet another alternative, one or more of the support straps 70, 80 can be releasably or refastenably attached to the elasticized support waistband 100, such as via cooperating hook and loop fastening components similar to those described earlier.

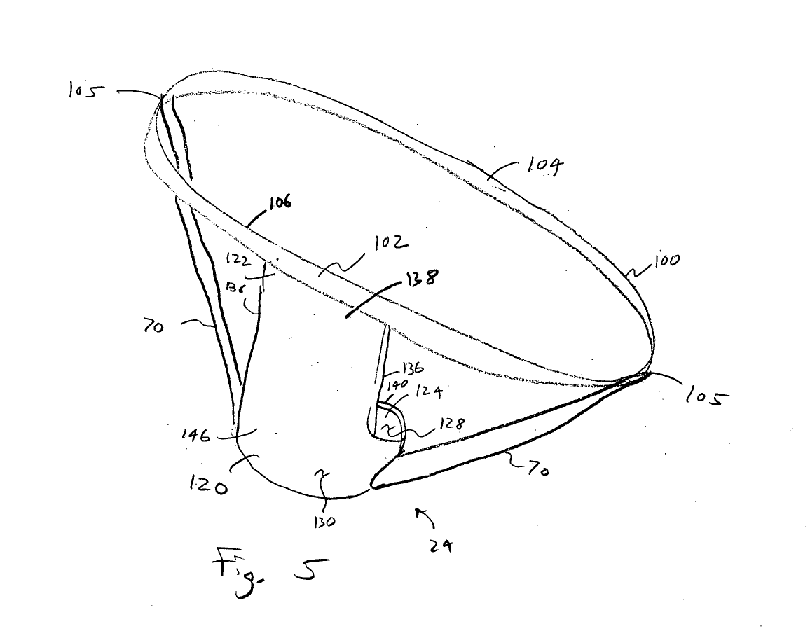

In those embodiments of the absorbent garment of the present invention that include an elasticized support waistband 100, and that further include one or more support straps 70, 80 (discussed above), the support straps can take any of the configurations previously described. For example, as representatively illustrated in FIG. 4, a single posterior support strap 70 can connect the absorbent composite 120 to the elasticized support waistband back waist section 104. The posterior support strap 70 can be wider than, narrower than, or the same width as the absorbent composite 120. In an alternative embodiment, the width of the at least one posterior support strap 70 increases as it travels in a direction from the absorbent composite 120 to the back waist section 104 of the elasticized support waistband 100. Such a configuration can provide added comfort to the wearer. For example, the width of a posterior support strap 70 can increase at least about twofold, more particularly at least about threefold, and still more particularly at least about fivefold, as it travels in a direction from the absorbent composite 120 to the back waist section 104 of the elasticized support waistband 100.

In alternative embodiments, the absorbent assembly 24 can include an elasticized support waistband 100 and two posterior support straps 70. For example, as representatively illustrated in FIGS. 2, 3, 5, and 8, two posterior support straps 70 can connect the absorbent composite 120 to at least one of the front waist section 102 and the back waist section 104 of the elasticized support waistband 100, including both the front and back waist sections 102, 104 at side waist sections 105 (generally, the regions where the front waist section 102 meets the back waist section 104) of the elasticized support waistband 100. In particular embodiments, again representatively illustrated in FIGS. 2, 3, 5, and 8, the two posterior support straps 70 diverge from each other as they travel in a direction from the absorbent composite 120 to the elasticized support waistband 100, such as to the side waist sections 105 of the elasticized support waistband 100. If two posterior support straps 70 are present and diverge from each other, they can, but need not, connect the back region 124 of the absorbent composite 120 to the front region 122 of the absorbent composite 120 (representatively illustrated in FIG. 10).

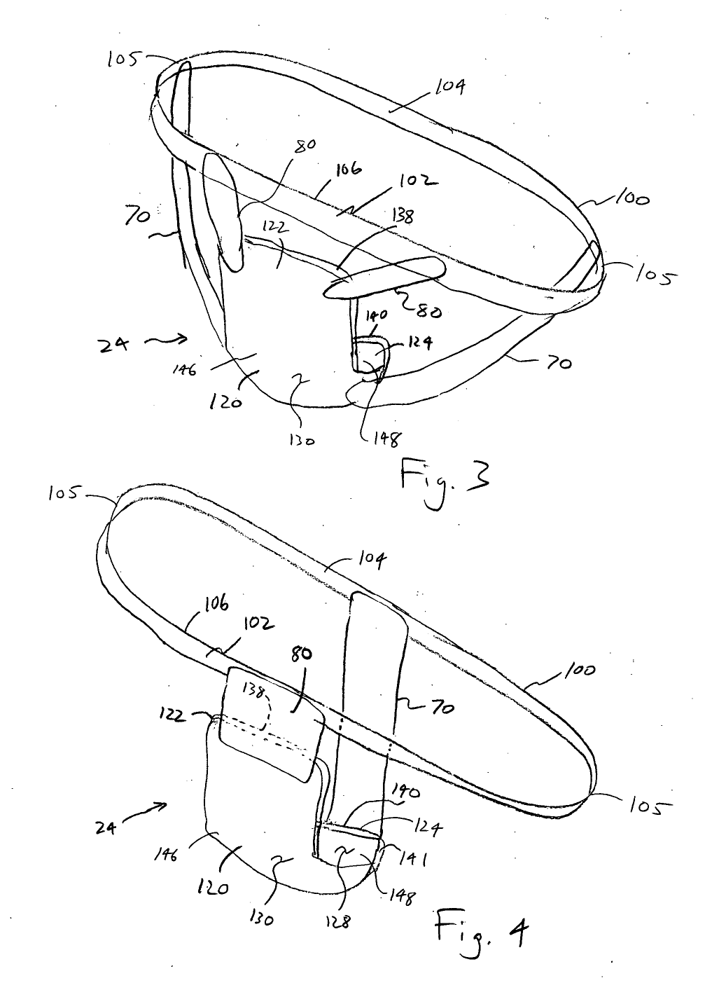

In particular embodiments, the absorbent assembly 24 further includes an elasticized support waistband 100 and at least one anterior support strap 80. For example, as representatively illustrated in FIG. 4, an anterior support strap 80 can extend from the absorbent composite front waist section 102 of the elasticized support waistband 100. The anterior support strap 80 can be wider than, narrower than, or the same width as the absorbent composite 120. In alternative embodiments, the absorbent assembly 24 can include an elasticized support waistband 100 and two anterior support straps 80. For example, as representatively illustrated in FIGS. 2 and 3, two anterior support straps 80 can connect the absorbent composite 120 to the front waist section 102 of the elasticized support waistband 100. In particular embodiments, again representatively illustrated in FIGS. 2 and 3, two anterior support straps 80 diverge from each other as they travel in a direction from the absorbent composite 120 to the front waist section 102 of the elasticized support waistband 100.

In particular embodiments, the elasticized support waistband 100 can be, but need not be, stretchable, and more suitably elastic. For example, the elasticized support waistband 100 can be stretchable to a length in the range of about 5 percent to about 75 percent of its unstretched length. The elasticized support waistband 100 is suitably constructed of elastomeric materials such as those described above as suitable for construction of the support straps 70, 80. In particular embodiments, the absorbent garment 10 of the present invention include both a garment shell 22 having waist elastic members 64 disposed thereon to created an elasticized shell waistband 60, and an elasticized support waistband 100 connected to the body-side surface 27 of the garment shell 22. In one such embodiment, the elasticized support waistband 100 is superposed over the elasticized shell waistband 60. In certain embodiments, waist elastic members 64 are affixed to the inside of the front and/or back waist regions 32, 34 of the garment shell, in which case the waist elastic members 64 partially define the body-side surface 27 of the garment shell 22.

It should be noted that the elasticized shell waistband 60, the elasticized support waistband 100, the posterior support straps 70, and the anterior support straps 80 can have the same elastic properties, or can have different elastic properties, in any combination. For example, in one embodiment, the elasticized support waistband 100 can exhibit a maximum elongation and/or tension (measured at 90% elongation) equal to, less than, or greater than the tension of the elasticized shell waistband 60. In a particular embodiment, the elasticized support waistband 100 exhibits a tension (measured at 90% elongation) at least about 10%, and more particularly at least about 20% greater than a tension exhibited by the elasticized shell waistband 60 (measured at 90% elongation). Such a configuration can, in certain embodiments, allow the absorbent assembly 24 to fit snugly against the body, while at the same time allowing the garment shell 22 to gather around the waist in a relatively gentle manner.

As previously mentioned, the elasticized support waistband 100 can, but need not, fully encircle the wearer. For example, the respective ends (not shown) of an elasticized support waistband could each be attached to upper corners of the absorbent composite 120 at the respective side edges 136 thereof. In other embodiments, such as those representatively illustrated in FIGS. 2-5 and 8, the elasticized support waistband 100 is configured to fully encircle a wearer.

In certain embodiments, the elasticized shell waistband 60 defines a shell waistband edge 61, and the elasticized support waistband 100 defines a support waistband edge 106. The shell waistband edge 61 and the support waistband edge 106 can be coterminous. In an alternative embodiment, the shell waistband edge 61 and the support waistband edge 106 are not coterminous, but are instead positioned with respect to each other such that the shell waistband edge 61 would be positioned higher on a wearer's waist than would the support waistband edge 106. In certain embodiments, the elasticized shell waistband 60 and the elasticized support waistband 100 do not overlap each other in the longitudinal direction. In other words, in certain embodiments, the elasticized shell waistband 60 and the elasticized support waistband 100 are positioned at different positions along the height of a standing wearer. It should be noted that the elasticized shell waistband 60 and the elasticized support waistband 100 can have the same longitudinal width, or can have different longitudinal widths.

Another aspect of the present invention is a package comprising at least one garment shell 22 and a plurality of inner absorbent assemblies 24. Each garment shell 22 and each inner absorbent assembly 24 can be configured in any of the ways described above, except that in the present aspect of the invention, each garment shell 22 and each absorbent assembly 24 is preferably (although not necessarily) constructed to allow for refastenable connection between them, respectively. In this way, a package including a plurality of absorbent assemblies 24, along with a lesser number of garment shells 22, can be sold. In such a scenario, each absorbent assembly 24 can be discarded following imbibement of a bodily fluid, but each of the at least one garment shells 22 can be reused with fresh absorbent assemblies. In one embodiment, each garment shell 22 and each inner absorbent assembly 24 are not configured to refastenably connect to each other. Instead, a wearer could simply position an absorbent assembly 24 on himself, such as one of the absorbent assemblies representatively illustrated in FIGS. 3-5 or 9-10, and subsequently position a garment shell 22 on himself.

The package can, but need not, include a packaging material. Packaging materials suitable for providing absorbent garments to consumers are well known in the art, and include such materials as boxes and bags. In one example, the packaging material is a polymeric bag.

As various changes could be made in the above constructions and methods, without departing from the scope of the invention, it is intended that all matter contained in the above description and shown in the accompanying drawings shall be interpreted as illustrative and not in a limiting sense.

When introducing elements of the invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

1. An absorbent garment comprising a garment shell and an inner absorbent assembly,
wherein the garment shell comprises a front panel assembly defining a front waist region, and a back panel assembly defining a back waist region, the front panel assembly being connected to the back panel assembly so as to define a waist opening and at least one leg opening, the garment shell further comprising an elasticized shell waistband, the elasticized shell waistband adapted to encircle the wearer, the garment shell defining a body-side surface and an outward surface;
wherein the inner absorbent assembly comprises:
an absorbent composite, the absorbent composite defining a front region and a back region;
an elasticized support waistband defining a front waist section and a back waist section, the front waist section being connected to the absorbent composite front region; and
at least one posterior support strap connecting the elasticized support waistband to the absorbent composite;
wherein the elasticized support waistband is connected to the body-side surface of the garment shell.

2. The absorbent garment of claim 1, wherein the elasticized shell waistband defines a shell waistband edge, and the elasticized support waistband defines a support waistband edge, and the shell waistband edge and the support waistband edge are coterminous.

3. The absorbent garment of claim 1, wherein the elasticized shell waistband defines a shell waistband edge, and the elasticized support waistband defines a support waistband edge, and the support waistband edge and the shell waistband edge are positioned with respect to each other such that the shell waistband edge would be positioned higher on a wearer's waist than would the support waistband edge.

4. The absorbent garment of claim 1, wherein the at least one posterior support strap is narrower in width than the absorbent composite.

5. The absorbent garment of claim 1, wherein the width of the posterior strap increases from the absorbent composite to the elasticized support waistband.

6. The absorbent garment of claim 1, wherein the absorbent assembly includes two posterior support straps which connect the elasticized support waistband to the absorbent composite.

7. The absorbent garment of claim 6, wherein the two posterior support straps diverge from each other from the absorbent composite to the elasticized support waistband.

8. The absorbent garment of claim 7, wherein the two posterior support straps are connected to the back region of the absorbent composite.

9. The absorbent garment of claim 1, wherein the elasticized support waistband is connected to the body-side surface of the garment shell at the front waist region of the garment shell.

10. The absorbent garment of claim 1, wherein the elasticized support waistband is connected to the body-side surface of the garment shell at both the front waist region and the back waist region of the garment shell.

11. The absorbent garment of claim 1, wherein the elasticized support waistband is releasably connected to the body-side surface of the garment shell.

12. The absorbent garment of claim 1, wherein the elasticized support waistband is refastenably connected to the body-side surface of the garment shell.

13. The absorbent garment of claim 1, further comprising at least one anterior support strap connecting the elasticized support waistband to the absorbent composite.

14. The absorbent garment of claim 13, wherein the anterior support strap is connected to the front region of the absorbent composite.

15. The absorbent garment of claim 1, wherein the at least one posterior support strap is elastomeric.

16. The absorbent garment of claim 1, wherein the garment also includes a crotch region interconnecting the front and back panels to define two leg openings.

17. The absorbent garment of claim 1, wherein the absorbent composite back region defines a back end and the absorbent composite front region defines a front end, and wherein the absorbent composite further defines two side edges, each side edge extending between the front and back end, and wherein the two side edges, the front end, and the back end are urged upward generally toward the wearer to define an upwardly urged periphery of the absorbent composite.

18. The absorbent garment of claim 17, wherein the upwardly urged periphery of the absorbent composite comprises absorbent material which provides at least about 20% of an absorbent capacity of the absorbent composite.

19. An absorbent garment comprising a garment shell and an inner absorbent assembly,
wherein the garment shell comprises a front panel assembly defining a front waist region, and a back panel assembly defining a back waist region, the front panel assembly being connected to the back panel assembly so as to define a waist opening and at least one leg opening, the garment shell further comprising an elasticized shell waistband, the elasticized shell waistband adapted to encircle the wearer, the garment shell defining a body-side surface and an outward surface;
wherein the inner absorbent assembly comprises:
an absorbent composite, the absorbent composite defining a front region and a back region, the front region being connected to the front waist region of the garment shell;
two posterior support straps which connect the absorbent composite to at least one of the front waist region and the back waist region of the garment shell, the two posterior support straps diverging from each other from the absorbent composite to the at least one of the front waist region and the back waist region.

20. The absorbent garment of claim 19, wherein the two posterior support straps are releaseably connected to the garment shell.

21. The absorbent garment of claim 19, wherein the two posterior support straps each include a frangible line of weakness to allow disconnection of the absorbent composite from the garment shell.

22. The absorbent garment of claim 19, wherein the absorbent composite front region is releaseably connected to the front waist region of the garment shell.

23. The absorbent garment of claim 19, further comprising at least one anterior support strap connecting the absorbent composite front region to the front waist region of the garment shell.

24. The absorbent garment of claim 23, wherein the at least one anterior support strap is releaseably connected to the front waist region of the garment shell.

25. The absorbent garment of claim 23, wherein the at least one anterior support strap includes a frangible line of weakness to allow disconnection of the absorbent composite from the front waist region of the garment shell.

26. The absorbent garment of claim 19, wherein the garment also includes a crotch region interconnecting the front and back panel assemblies to define two leg openings.

27. The absorbent garment of claim 19, wherein the absorbent composite back region defines a back end and the absorbent composite front region defines a front end, and wherein the absorbent composite further defines two side edges, each side edge extending between the front and back end, and wherein the two side edges, the front end, and the back end are urged upward generally toward the wearer to define an upwardly urged periphery of the absorbent composite.

28. The absorbent garment of claim 9, wherein the upwardly urged periphery of the absorbent composite comprises absorbent material which provides at least about 20% of an absorbent capacity of the absorbent composite.

29. An absorbent garment comprising a garment shell and an inner absorbent assembly,
wherein the garment shell comprises a front panel assembly defining a front waist region, and a back panel assembly defining a back waist region, the front panel assembly being connected to the back panel assembly so as to define a waist opening and at least one leg opening, the garment shell further comprising an elasticized shell waistband, the elasticized shell waistband adapted to encircle the wearer, the garment shell defining a body-side surface and an outward surface;
wherein the inner absorbent assembly comprises:
an absorbent composite, the absorbent composite defining a front region and a back region, the front region being connected to the front waist region of the garment shell; and
two posterior support straps which connect the front region of the absorbent composite to the back region of the absorbent composite, the two posterior support straps diverging from each other from the absorbent composite back region to the absorbent composite front region.

30. The absorbent garment of claim 1, further comprising an elasticized support waistband defining a front waist section and a back waist section, wherein the front waist section is connected to the absorbent composite front region, and wherein the elasticized support waistband is connected to the body-side surface of the garment shell.

31. An absorbent garment comprising a garment shell and an inner absorbent assembly,
wherein the garment shell comprises a front panel assembly defining a front waist region, and a back panel assembly defining a back waist region, the front panel assembly being connected to the back panel assembly so as to define a waist opening and at least one leg opening, the garment shell further comprising an elasticized shell waistband, the elasticized shell waistband adapted to encircle the wearer, the garment shell defining a body-side surface and an outward surface;
wherein the inner absorbent assembly comprises:
an absorbent composite, the absorbent composite defining a front region and a back region, the front region being connected to the front waist region of the garment shell; and
at least one posterior support strap which connects the absorbent composite to the back waist region of the garment shell.

32. The absorbent garment of claim 31, wherein the at least one posterior support strap is narrower in width than the absorbent composite.

33. The absorbent garment of claim 31, wherein the width of the at least one posterior strap increases from the absorbent composite to the back waist region of the garment shell.

34. The absorbent garment of claim 31, wherein the absorbent assembly is releasably connected to the body-side surface of the garment shell.

35. The absorbent garment of claim 31, wherein the absorbent assembly is refastenably connected to the body-side surface of the garment shell.

36. The absorbent garment of claim 31, further comprising at least one anterior support strap connecting the garment shell to the absorbent composite.

37. The absorbent garment of claim 31, wherein the at least one posterior support strap is elastomeric.

38. A package comprising at least one garment shell and a plurality of inner absorbent assemblies,
the at least one garment shell comprising a front panel assembly defining a front waist region, and a back panel assembly defining a back waist region, the front panel assembly being connected to the back panel assembly so as to define a waist opening and at least one leg opening, the garment shell further comprising an elasticized shell waistband, the elasticized shell waistband adapted to encircle the wearer, the garment shell defining a body-side surface and an outward surface;
each inner absorbent assembly comprising:
an absorbent composite, the absorbent composite defining a front region and a back region;
an elasticized support waistband defining a front waist section and a back waist section, the front waist section being connected to the absorbent composite front region; and
at least one posterior support strap connecting the elasticized support waistband to the absorbent composite.

39. The package of claim 38, wherein each absorbent assembly includes two posterior support straps which connect the elasticized support waistband to the absorbent composite.

40. The package of claim 39, wherein the two posterior support straps diverge from each other from the absorbent composite to the elasticized support waistband.

41. The package of claim 38, wherein the elasticized support waistband of each absorbent assembly is adapted to engage the body-side surface of the at least one garment shell at the front waist region thereof.
